(19) United States
(12) Patent Application Publication
(10) Pub. No.: US 2016/0060252 A1
(43) Pub. Date: Mar. 3, 2016

(54) 5-METHYLURIDINE METHOD FOR PRODUCING FESTINAVIR

(71) Applicant: BRISTOL-MYERS SQUIBB COMPANY, Princeton, NJ (US)

(72) Inventors: William P. Gallagher, Clarksburg, NJ (US); Prashant P. Deshpande, Princeton, NJ (US); Jun Li, Langhorne, PA (US); Kishta Katipally, Monmouth Junction, NJ (US)

(21) Appl. No.: 14/784,460

(22) PCT Filed: Apr. 14, 2014
(86) PCT No.: PCT/US14/33972

§ 371 Date: Oct. 14, 2015

Related U.S. Application Data
(60) Provisional application No. 61/812,345, filed on Apr. 16, 2013.

(51) Int. Cl. C07D 405/04; C07D 493/04

(52) U.S. Cl. CPC C07D 405/04; C07D 493/04

(57) ABSTRACT

The NRTI compound festinavir is made using 5-methyluridine as a starting material, followed by Claisen rearrangement.

### CROSS REFERENCE TO RELATED APPLICATION

This application claims the priority of U.S. Provisional Application Ser. No. 61/812,345 filed Apr. 16, 2013 which is herein incorporated in its entirety by reference.

### FIELD OF THE INVENTION

The present invention relates to one or more methods for producing the compound festinavir. More particularly, the invention is directed to an improved method for producing festinavir in good yield utilizing a different starting material and reaction mechanism(s) than has been used to date. The invention is also directed to festinavir and the intermediate compounds produced by the process(es) herein.

### BACKGROUND OF THE INVENTION

The compound known as festinavir is a nucleoside reverse transcriptase inhibitor (NRTI) which is being developed for the treatment of HIV infection. The drug has shown considerable efficacy in early development, and with perhaps less toxicity than some other NRTIs, such as the drug stavudine (marketed under the trade name ZERIT®). Festinavir has the chemical formula C₁₁N₂O₄H₈, and the structural formula:

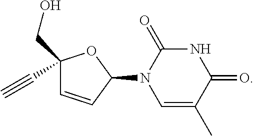 [SMILES: C#C[C@@]1(CO)C=C[C@H](n2cc(C)c(=O)[nH]c2=O)O1]

Festinavir was developed by Yale University in conjunction with two Japanese research scientists, and is protected by U.S. Pat. No. 7,589,078, the contents of which are incorporated herein by reference. The '078 patent sets forth the synthesis of the primary compound, and other structural analogs. In addition, Oncolys BioPharma, Inc. of Japan has now published US 2010/0280235 for the production of 4′ ethynyl D4T. As starting raw material, the Oncolys method utilizes a substituted furan compound, furfuryl alcohol. In another publication by Nissan Chemical Industries of Japan, and set forth in WO 2011/099443, there is disclosed a method for producing a beta-dihydrofuran deriving compound or a beta-tetrahydrofuran deriving compound. In this process, a diol compound is used as the starting material. Nissan has also published WO 2011/09442 directed to a process for the preparation of a β-glycoside compound. Two further publications, each to Hamari Chemicals of Japan, WO 2009/119785 and WO 2009/125841, set forth methods for producing and purifying ethynyl thymide compounds. Pharmaset, Inc. of the U.S. has also published US 2009/0318380, WO 2009/005674 and WO 2007/038507 for the production of 4′-nucleoside analogs for treating HIV infection. Reference is also made to the BMS application entitled “Sulfilimine and Sulphoxide Methods for Producing Festinavir” filed as a PCT application, PCT/US2013/042150 on May 22, 2013 (now WO2013/177243).

What is now needed in the art are new methods for the production of festinavir. The newly developed methods should be cost effective and obtain the final compound in relatively high yield, and should also utilize different starting material(s) and process mechanisms than what has been set forth in the published art, or has been otherwise available to the skilled artisan.

### SUMMARY OF THE INVENTION

According to a first embodiment, the invention is directed to a method for making the compound of Formula I

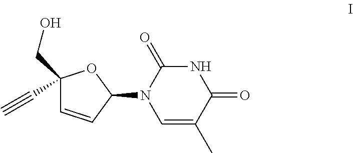 [SMILES: C#C[C@@]1(CO)C=C[C@H](n2cc(C)c(=O)[nH]c2=O)O1]

which comprises:

### (1) Treating 5-Methyluridine

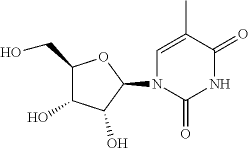 [SMILES: Cc1cn([C@@H]2O[C@H](CO)[C@@H](O)[C@H]2O)c(=O)[nH]c1=O]

with acid and acetaldehyde to produce the compound 1

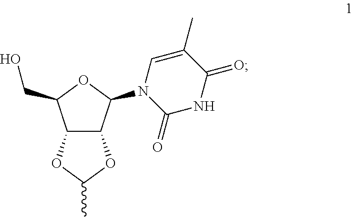

(2) Reacting compound 1 with 4-biphenyl acid chloride and pyridine in solvent to yield compound 2

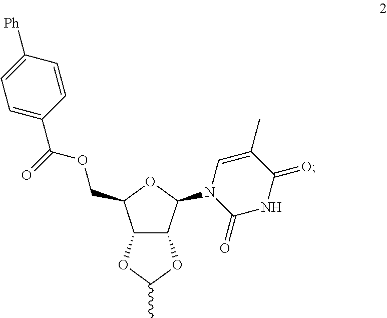

(3) Reacting compound 2 with a Lewis Acid and with triethylamine (Et₃N) in solvent, followed by reaction with aqueous acid or methanolic NH₄F, to produce compound 3

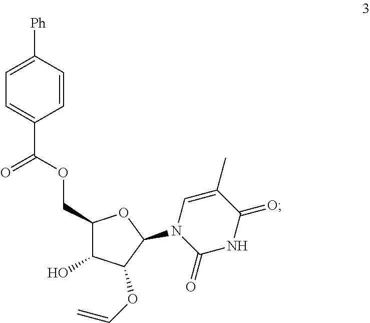 [SMILES: C=CO[C@@H]1[C@H](O)[C@@H](COC(=O)c2ccc(-c3ccccc3)cc2)O[C@H]1n1cc(C)c(=O)[nH]c1=O]

(4) Treating compound 3 with iodine (I₂), PPh₃ and imidazole with THF solvent to produce compound 4

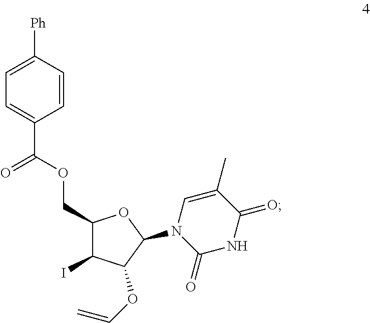 [SMILES: C=CO[C@@H]1[C@@H](I)[C@@H](COC(=O)c2ccc(-c3ccccc3)cc2)O[C@H]1n1cc(C)c(=O)[nH]c1=O]

(5) Performing iodide elimination reaction by heating a solution of compound 4 in toluene in the presence of a Lewis Base to yield compound 5

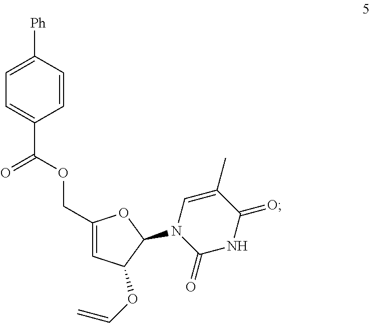 [SMILES: C=CO[C@@H]1C=C(COC(=O)c2ccc(-c3ccccc3)cc2)O[C@H]1n1cc(C)c(=O)[nH]c1=O]

(6) Conducting a Claisen rearrangement by heating compound 5 in a solvent with a boiling point over about 100° C. to produce compound 6

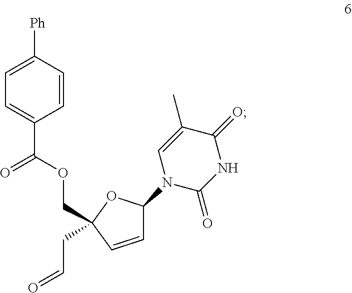 [SMILES: Cc1cn([C@H]2C=C[C@@](CC=O)(COC(=O)c3ccc(-c4ccccc4)cc3)O2)c(=O)[nH]c1=O]

(7) Reacting compound 6 with a TMSCl/Et₃N mixture, followed by NfF, and then warming in the presence of P-base to produce compound 7

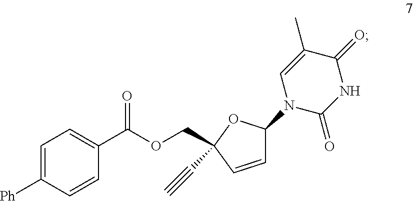 [SMILES: C#C[C@@]1(COC(=O)c2ccc(-c3ccccc3)cc2)C=C[C@H](n2cc(C)c(=O)[nH]c2=O)O1]

and
(8) Removing the ester protecting group by hydrolysis of compound 7 to yield the compound of Formula I.

In a further embodiment, the invention is also directed to festinavir and the intermediate compounds produced by the process(es) herein set forth.

The invention is directed to these, as well as to other important ends, hereinafter described.

### DETAILED DESCRIPTION OF THE EMBODIMENTS

For ease of reference, many reactants, reagents and solvents are set forth herein using their commonly accepted abbreviations.

The overall reaction scheme is presented below by way of example and illustration only, and should not be construed as limiting the scope of the invention:

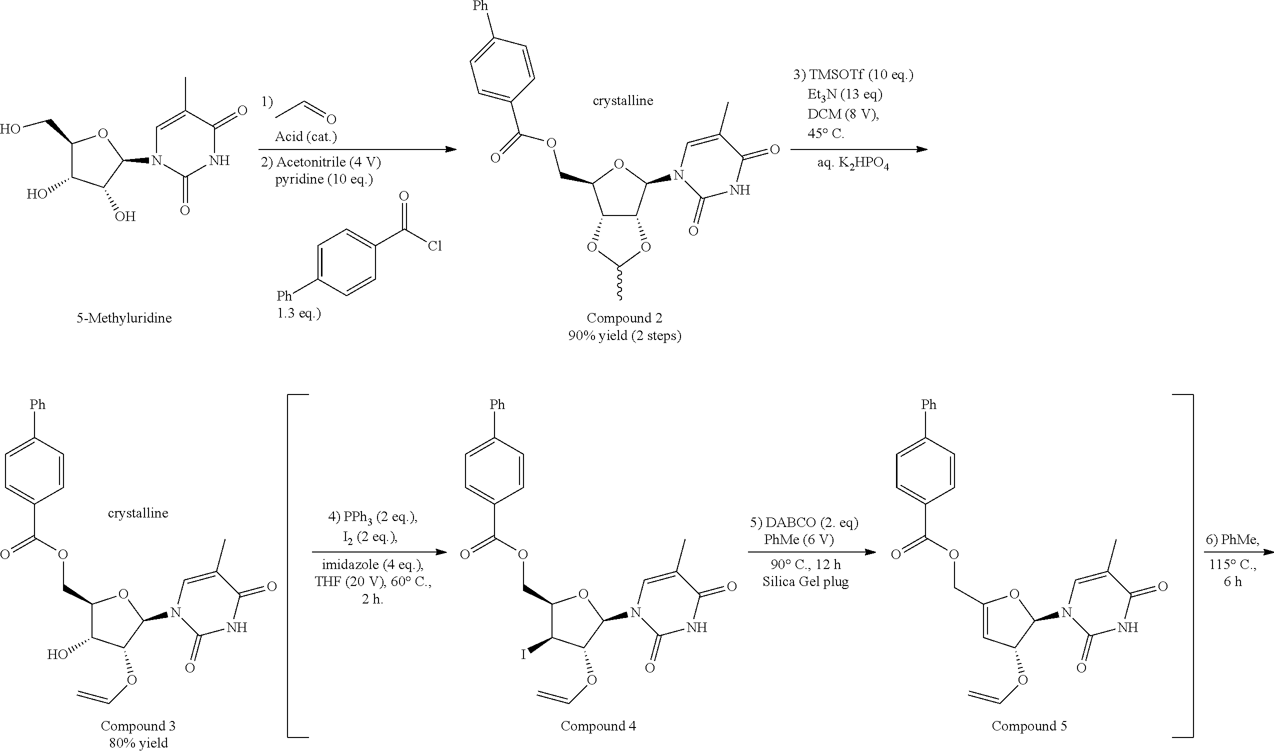

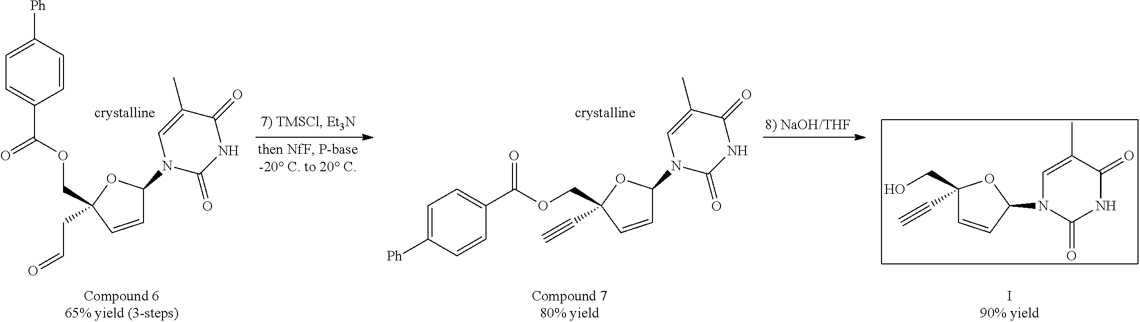 [SMILES: Cc1cn([C@H]2C=C[C@@](CC=O)(COC(=O)c3ccc(-c4ccccc4)cc3)O2)c(=O)[nH]c1=O ; C#C[C@@]1(COC(=O)c2ccc(-c3ccccc3)cc2)C=C[C@H](n2cc(C)c(=O)[nH]c2=O)O1 ; C#C[C@@]1(CO)C=C[C@H](n2cc(C)c(=O)[nH]c2=O)O1]

### Step#1: Acetal Formation

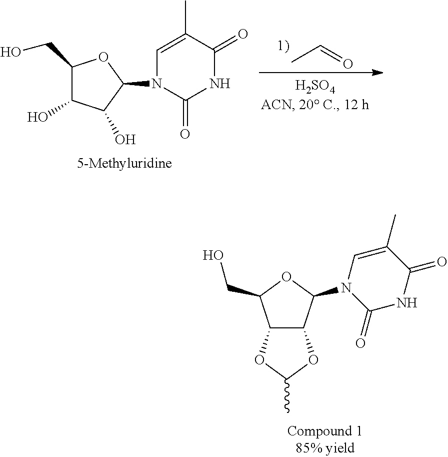

The starting material is 5-methylurdine, which is commercially available. The first step of the process is an acetal formation. 5-methyluridine is utilized and is treated with H₂SO₄ and acetaldehyde. Other acids available to the scientist, such as perchloric acid, will also work for this transformation. The solvent utilized for this step is acetonitrile (ACN), and other solvents may also be utilized as well. Once the starting material is consumed, a slurry is obtained and the product can be simply filtered off and dried to provide Compound 1 as a solid.

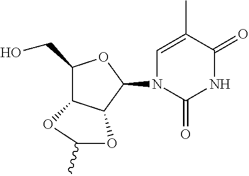

### Acetal Formation

### Preparation of 1-((3aR,4R,6R,6aR)-6-(hydroxymethyl)-2-methyltetrahydrofuro[3,4-d][1,3]dioxol-4-yl)-5-methylpyrimidine-2,4(1H,3H)-dione

The following were added to a flask: 5-methyluridine (10 g, 38.70 mmol), acetonitrile (20 mL) and 70% perchloric acid (4.01 mL, 47.63 mmol). A solution of acetaldehyde (3.26 mL, 58.10 mmol) in acetonitrile (20 mL) was added dropwise over 1 h. The resulting solution was allowed to stir at 20° C. for 18 h. The resulting slurry was filtered and dried (50° C., 25 mmHg) to afford Acetal (9.30 g, 84% yield) as white solid

¹H NMR (400 MHz, DMSO-d₆) δ=11.39 (s, 1H), 7.72-7.63 (m, 1H), 5.82 (d, J=3.0 Hz, 1H), 5.21-5.07 (m, 2H), 4.84 (dd, J=6.6, 2.5 Hz, 1H), 4.68 (dd, J=6.6, 3.0 Hz, 1H), 4.12-4.05 (m, 1H), 3.65-3.51 (m, 2H), 3.36 (s, 2H), 1.77 (s, 3H), 1.37 (d, J=5.1 Hz, 3H)

¹³C NMR (101 MHz, DMSO-d₆) 6=163.77, 150.32, 137.64, 109.39, 104.50, 90.79, 86.16, 83.83, 81.37, 61.25, 19.76, 12.06

### Step #2: Acetate Protection

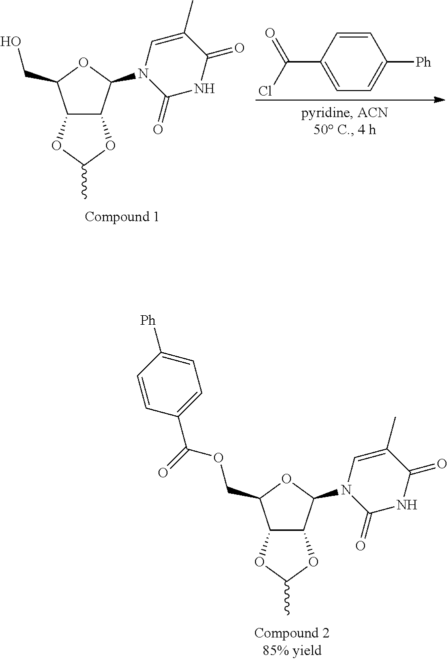

The next step of the sequence is installation of a 4-biphenylacetate. Without being bound by any particular theory, this protecting step may be chosen for two reasons:

1) To provide a solid intermediate that can be easily isolated, and

2) Act as a directing group in the next step (set forth later on).

This reaction consists of reacting Compound 1 with 4-biphenyl acid chloride and pyridine in acetonitrile. In this reaction, pyridine is preferred as it allows the reaction to occur only at the —OH moiety of the molecule. It should also be noted that other polar solvents could be used, but acetonitrile allowed the desired product Compound 2 to be isolated as s solid.

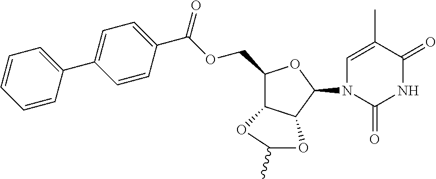

### Acylation

### Preparation of ((3aR,4R,6R,6aR)-2-methyl-6-(5-methyl-2,4-dioxo-3,4-dihydropyrimidin-1(2H)-yl)tetrahydrofuro[3,4-d][1,3]dioxol-4-yl)methyl [1,1′-biphenyl]-4-carboxylate

Acetal (9.30 g, 32 mmol) was dissolved into acetonitrile (100 mL). Pyridine (1.3 eq) was added followed by the addition of 4-biphenylcarbonyl chloride (1.05 eq). The solution was heated to 50° C. and held for 2 h. The slurry was cooled to 20° C. and held for 2 h. The slurry was filtered and washed with acetonitrile (100 mL). The solids were dried (50° C., 25 mmHg) to Compound 2 (85% yield).

¹H NMR (400 MHz, CHLOROFORM-d) δ=8.10 (d, J=8.1 Hz, 2H), 7.62 (d, J=7.6 Hz, 2H), 7.67 (d, J=8.1 Hz, 2H), 7.55-7.36 (m, 3H), 7.09 (s, 1H), 5.71 (s, 1H), 5.26 (q, J=4.7 Hz, 1H), 5.03 (dd, J=6.6, 2.0 Hz, 1H), 4.91 (dd, J=6.7, 3.2 Hz, 1H), 4.73-4.63 (m, 1H), 4.61-4.50 (m, 2H), 2.02 (s, 3H), 1.85-1.76 (m, 3H), 1.52 (d, J=4.8 Hz, 3H)

¹³C NMR (101 MHz, CHLOROFORM-d) δ=164.02, 161.94, 148.20, 144.18, 137.85, 135.89, 128.20, 127.05, 126.36, 126.30, 125.35, 125.26, 114.49, 109.20, 103.88, 92.51, 83.36, 83.29, 79.87, 75.45, 75.13, 74.81, 62.54, 17.92, 10.32, −0.01

### Step #3: Regioselective Acetal Opening

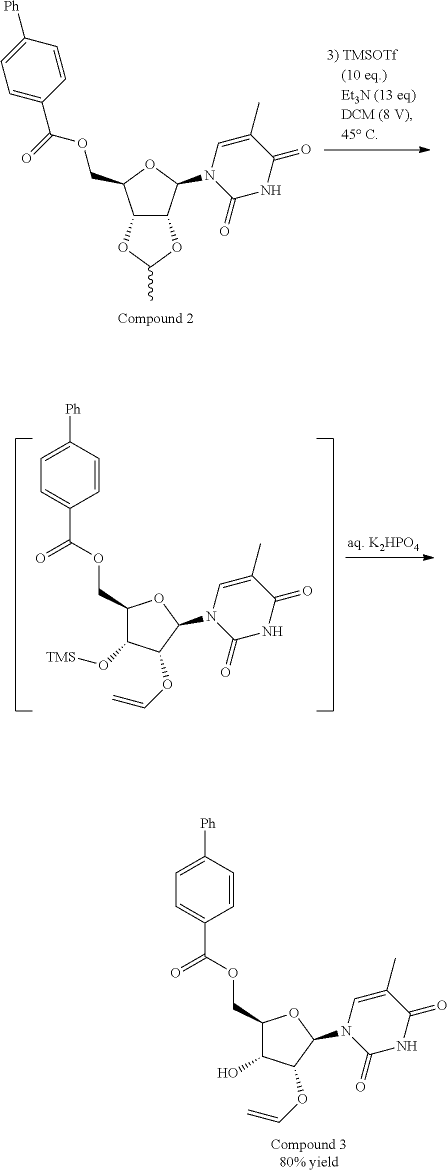

With the acetal and 4-biphenylacetate groups in place, the next reaction is a regioselective acetal opening utilizing TMSOTf (Trimethylsilyl trifluoromethane sulfonate, or other available Lewis acids)/Et₃N to afford the corresponding silyl ether, which is cleaved in situ, to afford the 2-vinyloxy compound as Compound 3. Compound 3 may be prepared in a step-wise fashion (shown below), but in order to reduce the number of steps, it is possible to take Compound 3 and selectively form the desired 2-vinyl oxy regioisomer Compound 3. Those skilled in the art may recognize that the 4-biphenylacetate can be important to obtain high selectivity for this transformation.

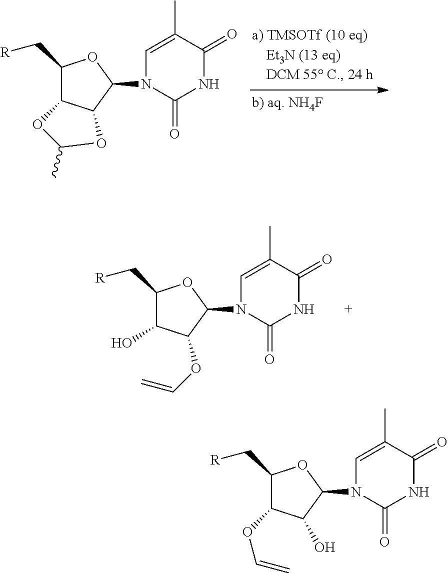

| Protecting Group | 2:3 |
|---|---|
| 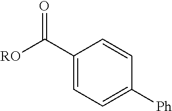 | 24:1 |
| 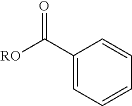 | 15:1 |
| 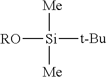 | 9:1 |
| 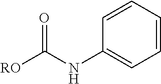 | 7:1 |
| Cl | 2:1 |
| 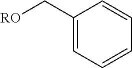 | 1:4 |

Although a variety of Lewis acids may be utilized, TMSOTf is generally found to be more effective. Et₃N is also a preferred reactant, as other amine bases are generally less effective. The ratio of TMSOTf to Et₃N is preferably within the range of about 1:1.3; if the reaction medium became acidic, Compound 3 would revert back to Compound 2. In terms of solvents, DCM (Dichloromethane) may be particularly effective, but toluene, CF₃-Ph, sulfolane, and DCE (Dichloroethene) are also effective. The reaction can be worked up using aqueous acid, preferably K₂HPO₄, or methanolic NH₄F to quench the reaction, as well as remove the TMS-ether in situ.

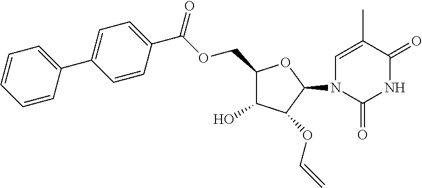 [SMILES: C=CO[C@@H]1[C@H](O)[C@@H](COC(=O)c2ccc(-c3ccccc3)cc2)O[C@H]1n1cc(C)c(=O)[nH]c1=O]

### TMSOTf-Opening

### Preparation of ((2R,3R,4R,5R)-3-hydroxy-5-(5-methyl-2,4-dioxo-3,4-dihydropyrimidin-1(2H)-yl)-4-(vinyloxy)tetrahydrofuran-2-yl)methyl [1,1′-biphenyl]-4-carboxylate

Compound 2 (20 g, 43.06 mmol) was dissolved into DCM (160 mL). Triethylamine (78 mL, 560 mmol) was added followed by the addition of TMSOTf (80.30 mL, 431 mmol). This solution was heated to 45° C. and held there until complete by HPLC analysis (6 h). Once complete, this solution was added to ammonium acetate (66.40 g, 861 mmol) in water (200 mL). After stirring for 20 min, the layers were separated. The organics were concentrated and the resulting residue was dissolved into EtOAc (200 mL). The organics were washed with the following solution (potassium phosphate monobasic (118 g, 861 mmol) in water (400 mL). The organics were then dried (Na₂SO₄), filtered and concentrated. The resulting residue was purified by column chromatography [Silica gel; 20% to 90% EtOAc in Hexanes] to afford Compound 3 (15.8 g, 79% yield) as a solid.

¹H NMR (400 MHz, CHLOROFORM-d) δ=9.18 (br. s., 1H), 8.18-8.06 (m, 2H), 7.73-7.56 (m, 4H), 7.55-7.38 (m, 3H), 7.24 (d, J=1.3 Hz, 1H), 6.59 (dd, J=14.0, 6.4 Hz, 1H), 5.81 (d, J=2.0 Hz, 1H), 4.84 (dd, J=12.6, 2.5 Hz, 1H), 4.63 (dd, J=12.5, 4.2 Hz, 1H), 4.59-4.44 (m, 3H), 4.40-4.26 (m, 2H), 1.70 (d, J=1.0 Hz, 3H)

¹³C NMR (101 MHz, CHLOROFORM-d) δ=166.13, 163.65, 150.00, 149.67, 146.39, 139.66, 135.67, 130.16, 129.01, 128.40, 128.06, 127.32, 127.28, 111.43, 91.93, 89.44, 81.60, 80.19, 69.32, 63.06, 12.32

### Step #4: Iodination

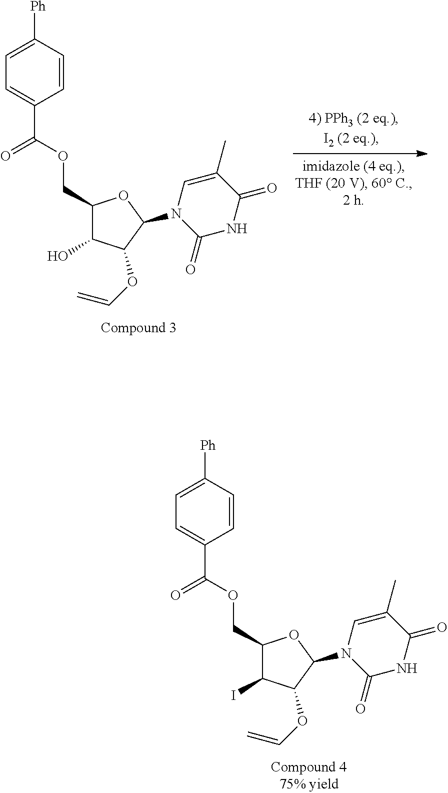 [SMILES: C=CO[C@@H]1[C@H](O)[C@@H](COC(=O)c2ccc(-c3ccccc3)cc2)O[C@H]1n1cc(C)c(=O)[nH]c1=O ; C=CO[C@@H]1[C@@H](I)[C@@H](COC(=O)c2ccc(-c3ccccc3)cc2)O[C@H]1n1cc(C)c(=O)[nH]c1=O]

Next, Compound 3 is transformed into the iodide compound which is Compound 4. This can be accomplished by treating Compound 3 with I₂(2.0 eq), PPh₃ (2.0 eq.) and imidazole (4.0 eq). Other methods to install the iodide may also be utilized, such as mesylation/NaI, etc., but these may be less preferred. In addition, other halogen-bearing compounds such as Br₂ and Cl₂ may be considered by the skilled scientist. Premixing imidazole, I₂, and PPh₃, followed by addition of Compound 3 in THF and heating at 60° C. allows smooth conversion to Compound 4. It is highly preferred to add all reagents prior to the addition of Compound 3; if not, the vinyloxy group will be cleaved. Other solvents, such as 2-MeTHF and PhMe may be utilized, but THF often provides the best yield.

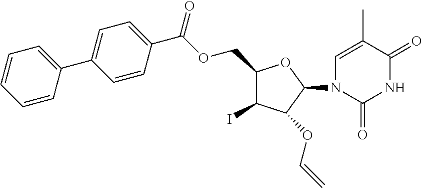 [SMILES: C=CO[C@@H]1[C@@H](I)[C@@H](COC(=O)c2ccc(-c3ccccc3)cc2)O[C@H]1n1cc(C)c(=O)[nH]c1=O]

### Iodination

### Preparation of ((2R,3S,4S,5R)-3-iodo-5-(5-methyl-2,4-dioxo-3,4-dihydropyrimidin-1(2H)-yl)-4-(vinyloxy)tetrahydrofuran-2-yl)methyl [1,1′-biphenyl]-4-carboxylate

The following were added to a flask: imidazole (8.79 g, 129 mmol), triphenylphosphine (16.94 g, 65 mmol), iodine 16.39 g, 65 mmol) and THF (525 mL). A solution of Compound 3 (15 g, 32 mmol) in THF (375 mL) was added. The solution was heated to 60° C. and was held at 60° C. for 4 h. Once complete by HPLC analysis (4 h), the solution was concentrated and the residue was purified by column chromatography [Silica gel; 10% to 60% EtOAc in Hexanes] to afford Compound 4 (17.0 g, 92% yield) as a solid.

¹H NMR (400 MHz, CHLOROFORM-d) δ=9.25 (br. s., 1H), 8.16 (d, J=8.3 Hz, 2H), 7.75-7.61 (m, 5H), 7.54-7.40 (m, 3H), 7.32-7.24 (m, 2H), 7.23-7.16 (m, 2H), 6.56-6.45 (m, 1H), 6.06 (d, J=1.5 Hz, 1H), 4.89 (s, 1H), 4.66 (dd, J=12.0, 6.9 Hz, 1H), 4.56 (dd, J=12.0, 3.9 Hz, 1H), 4.46 (d, J=4.0 Hz, 1H), 4.39-4.26 (m, 2H), 4.13 (dt, J=7.1, 3.8 Hz, 1H), 2.06-1.97 (m, 3H)

¹³C NMR (101 MHz, CHLOROFORM-d) δ=165.96, 163.94, 150.27, 149.29, 146.28, 139.81, 137.88, 135.84, 130.37, 129.06, 129.01, 128.34, 128.25, 127.94, 127.31, 127.22, 125.32, 111.07, 91.37, 90.32, 89.18, 78.43, 69.15, 25.81, 21.49, 12.71

### Step #5: Iodide Elimination

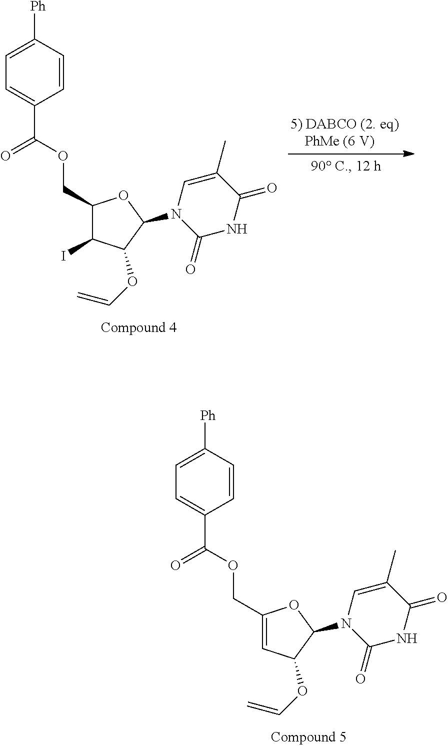 [SMILES: C=CO[C@@H]1[C@@H](I)[C@@H](COC(=O)c2ccc(-c3ccccc3)cc2)O[C@H]1n1cc(C)c(=O)[nH]c1=O ; C=CO[C@@H]1C=C(COC(=O)c2ccc(-c3ccccc3)cc2)O[C@H]1n1cc(C)c(=O)[nH]c1=O]

The next step of the sequence is to install the allyic moiety. Heating a solution of Compound 4 in toluene in the presence of DABCO (1,4-Diazabicyclo[2.2.2]octane) allows for elimination of the iodide. Other solvents, such as THF and DCE may be utilized, but toluene often provides the best conversion and yield. Other amine bases may be used in this transformation, but generally DABCO is preferred.

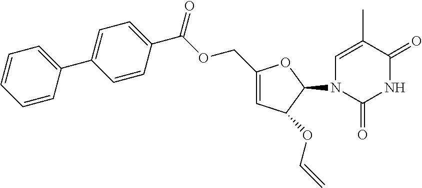 [SMILES: C=CO[C@@H]1C=C(COC(=O)c2ccc(-c3ccccc3)cc2)O[C@H]1n1cc(C)c(=O)[nH]c1=O]

### Elimination

### Preparation of ((4R,5R)-5-(5-methyl-2,4-dioxo-3,4-dihydropyrimidin-1(2H)-yl)-4-(vinyloxy)-4,5-dihydrofuran-2-yl)methyl [1,1′-biphenyl]-4-carboxylate

Compound 4 (17 g, 30 mmol) was dissolved into toluene (255 mL), and DABCO (10 g, 89 mmol) was added. The solution was heated to 90° C. and held there for 2 h. Once complete, the organics were washed with sat. aq. Na₂S₂O₃ (200 mL). The organics were then dried (Na₂SO₄), filtered, and concentrated. The resulting residue was purified by column chromatography [Silica gel; 5% to 60% EtOAc in Hexanes] to yield Compound 5 (10.9, 85% yield) as a foam.

¹H NMR (400 MHz, CHLOROFORM-d) δ=8.93 (br. s., 1H), 8.18-8.11 (m, 2H), 7.75-7.61 (m, 5H), 7.55-7.39 (m, 4H), 6.95 (d, J=1.0 Hz, 1H), 6.54 (d, J=2.0 Hz, 1H), 6.46 (dd, J=14.3, 6.7 Hz, 1H), 5.53 (d, J=2.5 Hz, 1H), 5.09 (d, J=2.8 Hz, 1H), 5.04 (d, J=6.6 Hz, 2H), 4.29 (dd, J=14.3, 2.4 Hz, 1H), 4.23 (dd, J=6.7, 2.4 Hz, 1H), 1.88 (d, J=1.0 Hz, 3H)

¹³C NMR (101 MHz, CHLOROFORM-d) δ=165.73, 159.58, 149.10, 146.49, 139.70, 134.51, 132.17, 132.07, 131.94, 131.92, 130.30, 129.01, 128.56, 128.44, 128.40, 127.73, 127.30, 127.28, 112.50, 99.16, 90.57, 90.23, 84.81, 58.68, 12.44

### Step #6: Claisen Rearrangement

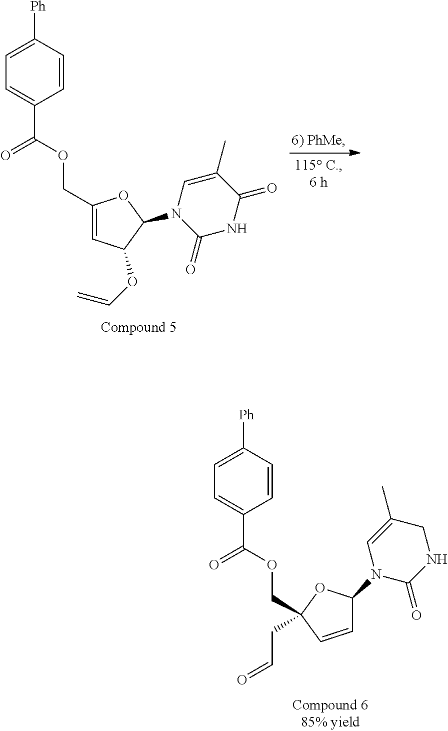 [SMILES: C=CO[C@@H]1C=C(COC(=O)c2ccc(-c3ccccc3)cc2)O[C@H]1n1cc(C)c(=O)[nH]c1=O ; CC1=CN([C@H]2C=C[C@@](CC=O)(COC(=O)c3ccc(-c4ccccc4)cc3)O2)C(=O)NC1]

An important reaction in the sequence is the Claisen rearrangement. This reaction is utilized to install the quaternary stereocenter and the olefin geometry in the ring. Heating Compound 5 in benzonitrile at 190° C. for 2-3 hours allows for smooth conversion to Compound 6, and after chromatography, a 90% yield can be achieved. Toluene (110° C., 8 h) also works to provide the desired Compound 6 as a solid by simply cooling the reaction to 20° C. (no chromatography). Other solvents with boiling points over about 100° C. may also be utilized.

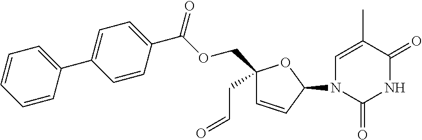 [SMILES: Cc1cn([C@H]2C=C[C@@](CC=O)(COC(=O)c3ccc(-c4ccccc4)cc3)O2)c(=O)[nH]c1=O]

### Claisen Rearrangement

### Preparation of ((2S,5R)-5-(5-methyl-2,4-dioxo-3,4-dihydropyrimidin-1(2H)-yl)-2-(2-oxoethyl)-2,5-dihydrofuran-2-yl)methyl [1,1′-biphenyl]-4-carboxylate

Compound 5 (1 mmol) was dissolved into benzonitrile (10 mL). The solution was heated to 190° C. for 3 h. After cooling to 20° C., the solution was purified by column chromatography [silica gel, 50:50 Hexanes:EtOAc] to afford Compound 6 (1 mmol).

Alternatively, Compound 5 (1 mmol) was dissolved into toluene (10 mL). The solution was heated to 110° C. and held for 12 h. Upon cooling to 20° C., a slurry formed. The solids were filtered, washed (PhMe) and dried (50° C., 25 mmHg) to afford Compound 6 (1 mmol) as a white solid.

¹H NMR (400 MHz, CHLOROFORM-d) δ=9.84 (t, J=1.8 Hz, 1H), 8.53 (br. s., 1H), 8.13-8.03 (m, J=8.3 Hz, 2H), 7.73-7.67 (m, 2H), 7.67-7.60 (m, 2H), 7.56-7.38 (m, 3H), 7.14 (d, J=1.3 Hz, 1H), 7.04 (t, J=1.5 Hz, 1H), 6.57 (dd, J=6.1, 2.0 Hz, 1H), 6.02 (dd, J=5.9, 1.1 Hz, 1H), 4.68-4.52 (m, 2H), 3.06-2.89 (m, 2H), 1.59 (d, J=1.0 Hz, 3H)

¹³C NMR (101 MHz, CHLOROFORM-d) δ=198.33, 165.83, 163.35, 150.65, 146.56, 139.63, 136.24, 135.02, 130.21, 129.04, 128.44, 127.86, 127.49, 127.41, 127.28, 111.59, 90.03, 89.61, 67.33, 50.06, 12.06

### Step #7: Alkyne Formation Via Elimination of Enol Nonaflate

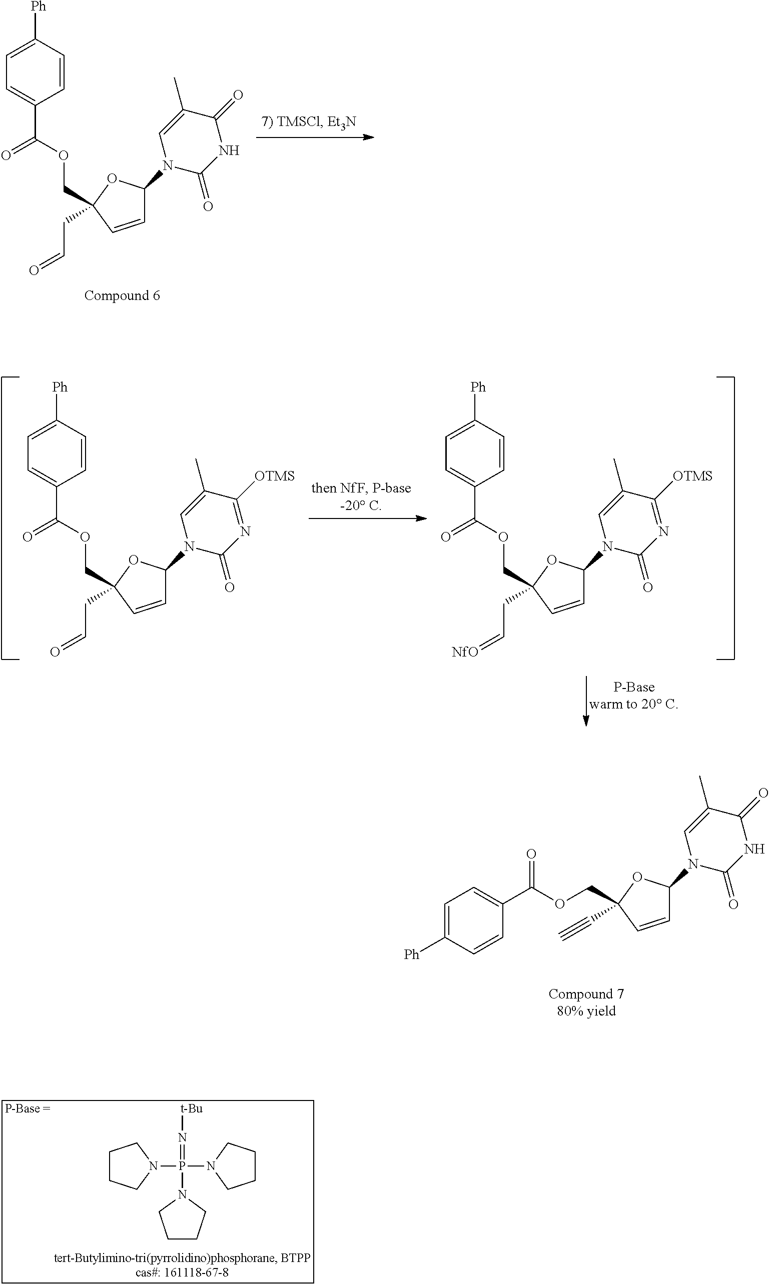

The alkyne formation is performed by first treating Compound 6 with TMSCl (Trimethylsilyl chloride)/Et₃N. NfF (Nonafluoro-1-butanesulfonyl fluoride) and P-base ( ) are then added at −20° C. After warming to 20° C., the desired alkyne Compound 7 can be isolated in about 80% yield. Initially, TMSCl is presumed to react at the NH moiety. NfF/P-base then reacts with the aldehyde to form the enol Nonaflate. Upon warming to 20° C. in the presence of P-base, the enol Nonaflate eliminates smoothly to the alkyne Compound 7. Without the TMSCl/Et₃N, the yields are only ˜25%.

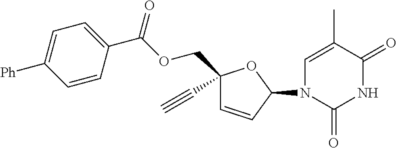 [SMILES: C#C[C@@]1(COC(=O)c2ccc(-c3ccccc3)cc2)C=C[C@H](n2cc(C)c(=O)[nH]c2=O)O1]

### Alkyne Formation

### Preparation of ((2R,5R)-2-ethynyl-5-(5-methyl-2,4-dioxo-3,4-dihydropyrimidin-1(2H)-yl)-2,5-dihydrofuran-2-yl)methyl [1,1′-biphenyl]-4-carboxylate

Compound 6 (1 g, 2.24 mmol) was dissolved into DMF (Dimethylformamide) (5 mL). (Other polar solvents could also have been used.) Triethylamine (406 uL, 2.91 mmol) was added and the solution was cooled to 0° C. TMSCl (314 uL, 2.46 mmol) was added and the solution was allowed to stir at 0° C. for 30 min. The solution was then cooled to −20° C., and NfF (484 uL, 2.69 mmol) was added and the solution was allowed to stir at −20° C. for 5 min. Phosphazane P1-base (1.54 mL, 4.93 mmol) was added dropwise over 20 min. The solution was then allowed to warm to 20° C. and held for 20 h. The solution was then poured into water (50 mL) and extracted with DCM (100 mL). The organics were concentrated and the resulting residue was purified by column chromatography [Silica gel; 10% to 60% EtOAc in Hexanes] to afford Compound 7 (816 mg, 85% yield) as a solid.

¹H NMR (400 MHz, DMSO-d₆) δ=11.46 (s, 1H), 8.08-7.97 (m, J=8.6 Hz, 2H), 7.92-7.80 (m, 2H), 7.73 (d, J=7.1 Hz, 2H), 7.59-7.39 (m, 3H), 7.06 (d, J=1.0 Hz, 1H), 6.89 (d, J=1.5 Hz, 1H), 6.61 (dd, J=5.6, 2.0 Hz, 1H), 6.23 (dd, J=5.6, 1.0 Hz, 1H), 4.66 (d, J=12.1 Hz, 1H), 4.57 (d, J=11.6 Hz, 1H), 3.87 (s, 1H), 1.37 (s, 3H)

¹³C NMR (101 MHz, DMSO-d₆) δ=164.89, 163.57, 150.61, 145.13, 138.73, 135.30, 134.40, 129.94, 129.12, 128.49, 127.84, 127.78, 127.18, 126.98, 110.01, 89.37, 83.69, 80.01, 78.23, 66.89, 11.46

### Step #8: Aromatic Ester Removal

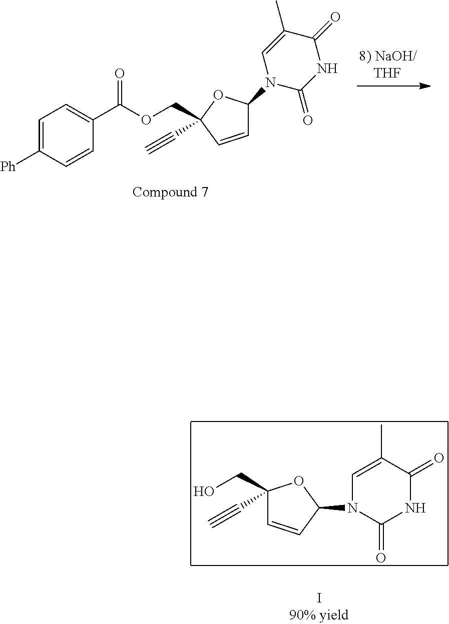 [SMILES: C#C[C@@]1(COC(=O)c2ccc(-c3ccccc3)cc2)C=C[C@H](n2cc(C)c(=O)[nH]c2=O)O1 ; C#C[C@@]1(CO)C=C[C@H](n2cc(C)c(=O)[nH]c2=O)O1]

The final step of the sequence is to remove the aromatic ester protecting group. This consists of hydrolysis by NaOH in aq. THF solution. The API is extracted into THF and then crystallized from THF/PhMe.

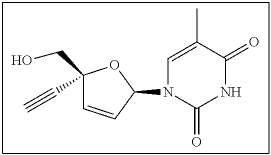 [SMILES: C#C[C@@]1(CO)C=C[C@H](n2cc(C)c(=O)[nH]c2=O)O1]

### Deprotection

### Preparation of 1-((2R,5R)-5-ethynyl-5-(hydroxymethyl)-2,5-dihydrofuran-2-yl)-5-methylpyrimidine-2,4(1H,3H)-dione (Ed4T)

Compound 7 (10 g, 23.40 mmol) was dissolved into THF (100 mL). 3N NaOH (10 mL) was added. The solution was allowed to stir at 20° C. for 12 h. The layers were split and the organics were kept. The organics were concentrated to reach a KF<1 wt %. Toluene (100 mL) was added, and solids crashed out of solution. The solids were filtered and washed with Toluene (100 mL). The solids were then dried (50° C., 25 mmHg) to afford Festinavir (5.21 g, 90% yield) as a white solid.

¹H NMR (400 MHz, DMSO-d₆) δ=11.36 (s, 1H), 7.58 (s, 1H), 6.89 (s, 1H), 6.36 (d, J=6.1 Hz, 1H), 6.05 (d, J=6.1 Hz, 1H), 5.48 (t, J=5.6 Hz, 1H), 3.78-3.49 (m, 3H), 3.46-3.31 (m, 1H), 1.71 (s, 3H)

¹³C NMR (101 MHz, DMSO-d₆) δ=163.80, 150.76, 136.75, 135.47, 127.06, 108.98, 88.87, 86.52, 81.37, 77.33, 65.68, 12.17.

The foregoing description is merely illustrative and should not be understood to limit the scope or underlying principles of the invention in any way. Indeed, various modifications of the invention, in addition to those shown and described herein, will become apparent to those skilled in the art from the foregoing description and examples. Such modifications are also intended to fall within the scope of the appended claims.

What is claimed is:

1. A method for making the compound of Formula I 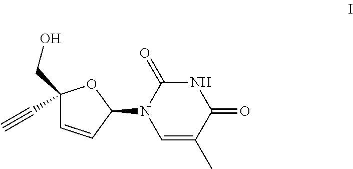 [SMILES: C#C[C@@]1(CO)C=C[C@H](n2cc(C)c(=O)[nH]c2=O)O1]
which comprises:
(1) Treating 5-Methyluridine 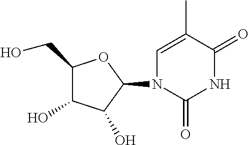 [SMILES: Cc1cn([C@@H]2O[C@H](CO)[C@@H](O)[C@H]2O)c(=O)[nH]c1=O]
with acid and acetaldehyde to produce the compound 1 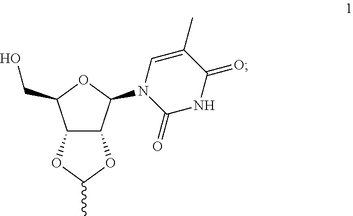
(2) Reacting compound 1 with 4-biphenyl acid chloride and pyridine in solvent to yield compound 2 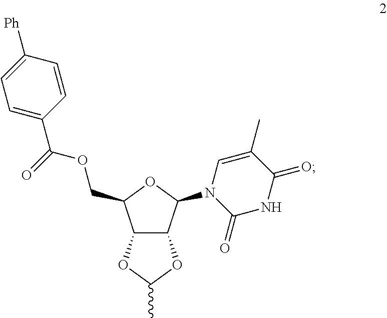
(3) Reacting compound 2 with Lewis acid and triethylamine (Et₃N) in solvent, followed by reaction with aqueous acid or methanolic NH₄F, to produce compound 3 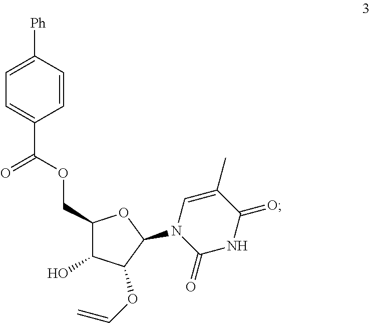 [SMILES: C=CO[C@@H]1[C@H](O)[C@@H](COC(=O)c2ccc(-c3ccccc3)cc2)O[C@H]1n1cc(C)c(=O)[nH]c1=O]
(4) Treating compound 3 with iodine (I₂), PPh₃ and imidazole with THF solvent to produce compound 4 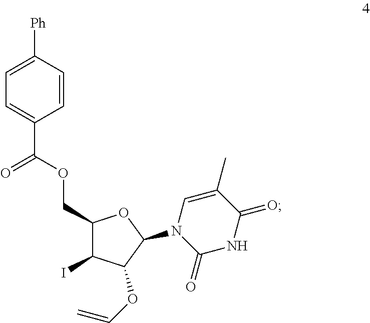 [SMILES: C=CO[C@@H]1[C@@H](I)[C@@H](COC(=O)c2ccc(-c3ccccc3)cc2)O[C@H]1n1cc(C)c(=O)[nH]c1=O]
(5) Performing iodide elimination reaction by heating a solution of compound 4 in toluene in the presence of a Lewis base to yield compound 5 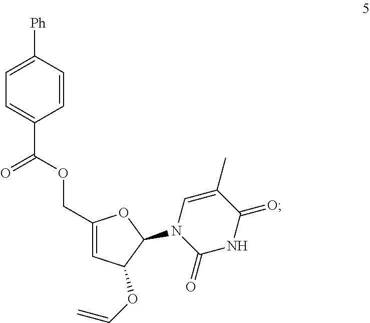 [SMILES: C=CO[C@@H]1C=C(COC(=O)c2ccc(-c3ccccc3)cc2)O[C@H]1n1cc(C)c(=O)[nH]c1=O]
(6) Conducting a Claisen rearrangement by heating compound 5 in benzonitrile or toluene to produce compound 6 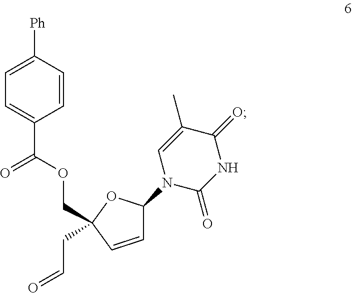 [SMILES: Cc1cn([C@H]2C=C[C@@](CC=O)(COC(=O)c3ccc(-c4ccccc4)cc3)O2)c(=O)[nH]c1=O]
(7) Reacting compound 6 with a TMSCl/Et₃N mixture, followed by NfF, and then warming in the presence of P-base to produce compound 7 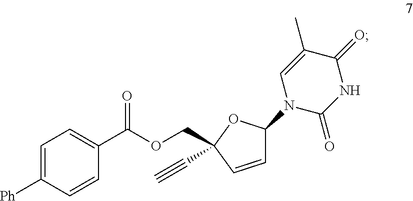 [SMILES: C#C[C@@]1(COC(=O)c2ccc(-c3ccccc3)cc2)C=C[C@H](n2cc(C)c(=O)[nH]c2=O)O1]
and
(8) Removing the ester protecting group by hydrolysis of compound 7 to yield the compound of Formula I.

2. The method of claim 1, wherein said acid in step (1) is selected from sulfuric and perchloric acids.

3. The method of claim 2, wherein said step (1) is conducted using acetonitrile as a solvent.

4. The method of claim 1, wherein said solvent in step (2) is acetonitrile or other polar solvents.

5. The method of claim 1, wherein said solvent in step (3) is selected from DCM, DCE, CF₃-Ph, toluene, and sulfolane.

6. The method of claim 5, wherein said solvent is DCM.

7. The method of claim 5, wherein said Lewis acid is TMSOTf.

8. The method of claim 1, wherein in step (5) said Lewis base is DABCO.

9. The method of claim 8, further comprising heating the mixture to about 60° C.

10. The method of claim 1, wherein said Claisen rearrangement of step (6) involves heating said compound 5 in benzonitrile at about 190° C. for about 2-3 hours.

11. The method of claim 1, wherein said Claisen rearrangement of step (6) involves heating said compound 5 in toluene at about 110° C. for about 8 hours.

12. The method of claim 1, wherein said compound 6 in step (7) is dissolved into DMF to form a solution, followed by addition of said triethylamine to said solution, and then said TMSCl.

13. The method of claim 12, wherein said NfF is added to said solution, followed by said P-1 base.

14. The method of claim 1, wherein said hydrolysis of compound 7 in step (8) is performed using sodium hydroxide (NaOH) in THF solution.

15. The method of claim 14, wherein said step (8) results in at least about 90% yield of compound 8 from compound 7.

16. The method of claim 1, wherein said aqueous acid in step (3) is K₂HPO₄.

17. Festinavir which is produced according to the process of claim 1.
